SEQUENCE LISTING (2 sequences)

SEQ ID NO: 1 - protein, 1464 aa, Homo sapiens [sequence omitted]

SEQ ID NO: 2 - protein, 1464 aa, Mus macedonicus
MGRLGYWTLLVLPALLVWHGPAQNAAAEKGTPALNIAVLLGHSHDVTERELRNLWGPEQATGLPLDVNVVALLMNRTDPKSLITHVCDLMSGARIHGLVFGDDTDQEAVAQMLDFISSQTFIPILGIHGGASMIMADKDPTSTFFQFGASIQQQATVMLKIMQDYDWHVFSLVTTIFPGYRDFISFIKTTVDNSFVGWDMQNVITLDTSFEDAKTQVQLKKIHSSVILLYCSKDEAVLILSEARSLGLTGYDFFWIVPSLVSGNTELIPKEFPSGLISVSYDDWDYSLEARVRDGLGILTTAASSMLEKFSYIPEAKASCYGQTEKPETPLHTLHQFMVNVTWDGKDLSFTEEGYQVHPRLVVIVLNKDREWEKVGKWENQTLSLRHAVWPRYKSFSDCEPDDNHLSIVTLEEAPFVIVEDIDPLTETCVRNTVPCRKFVKINNSTNEGMNVKKCCKGFCIDILKKLSRTVKFTYDLYLVTNGKHGKKVNNVWNGMIGEVVYQRAVMAVGSLTINEERSEVVDFSVPFVETGISVMVSRSNGTVSPSAFLEPFSASVWVMMFVMLLIVSAIAVFVFEYFSPVGYNRNLAKGKAPHGPSFTIGKAIWLLWGLVFNNSVPVQNPKGTTSKIMVSVWAFFAVIFLASYTANLAAFMIQEEFVDQVTGLSDKKFQRPHDYSPPFRFGTVPNGSTERNIRNNYPYMHQYMTKFNQRGVEDALVSLKTGKLDAFIYDAAVLNYKAGRDEGCKLVTIGSGYIFATTGYGIALQKGSPWKRQIDLALLQFVGDGEMEELETLWLTGICHNEKNEVMSSQLDIDNMAGVFYMLAAAMALSLITFIWEHLFYWKLRFCFTGVCSDRPGLLFSISRGIYSCIHGVHIEEKKKSPDFNLTGSQSNMLKLLRSAKNISNMSNMNSSRMDSPKRAADFIQRGSLIVDMVSDKGNLIYSDNRSFQGKDSIFGENMNELQTFVANRHKDSLSNYVFQGQHPLTLNESNPNTVEVAVSTESKGNSRPRQLWKKSMESLRQDSLNQNPVSQRDEKTAENRTHSLKSPRYLPEEVAHSDISETSSRATCHREPDNNKNHKTKDNFKRSMASKYPKDCSEVERTYVKTKASSPRDKIYTIDGEKEPSFHLDPPQFIENIVLPENVDFPDTYQDHNENFRKGDSTLPMNRNPLHNEDGLPNNDQYKLYAKHFTLKDKGSPHSEGSDRYRQNSTHCRSCLSNLPTYSGHFTMRSPFKCDACLRMGNLYDIDEDQMLQETGNPATREEAYQQDWSQNNALQFQKNKLKINRQHSYDNILDKPREIDLSRPSRSISLKDRERLLEGNLYGSLFSVPSSKLLGNKSSLFPQGLEDSKRSKSLLPDHTSDNPFLHTYGDDQRLVIGRCPSDPYKHSLPSQAVNDSYLRSSLRSTASYCSRDSRGHSDVYISEHVMPYAANKNNMYSTPRVLNSCSNRRVYKKMPSIESDV

(19) United States
(12) Patent Application Publication
(10) Pub. No.: US 2026/0118363 A1
(43) Pub. Date: Apr. 30, 2026

(54) Application of GLUN2A and NMDA Receptor Containing GLUN2A as Targets in Screening Drugs for Treating Depression

(71) Applicant: Synphatec (Shanghai) Biopharmaceutical Technology Co., Ltd., Shanghai (CN)

(72) Inventors: Yelin Chen, Shanghai (CN); Tonghui Su, Shanghai (CN); Yi Lu, Shanghai (CN); Chaoying Fu, Shanghai (CN); Yang Geng, Shanghai (CN)

(21) Appl. No.: 18/294,715

(22) PCT Filed: Jul. 15, 2022
(86) PCT No.: PCT/CN2022/106052

§ 371 Date: Feb. 2, 2024

(30) Foreign Application Priority Data
Aug. 6, 2021 (CN) 202110901806.8

(51) Int. Cl. G01N 33/68; A61K 31/165; A61K 31/675; A61K 31/7088; C07K 16/00; G01N 33/50

(52) U.S. Cl. CPC G01N 33/68; A61K 31/165; A61K 31/675; A61K 31/7088; C07K 16/00; G01N 33/5058; G01N 33/5088; G01N 2500/02; G01N 2500/04; G01N 2500/10

(57) ABSTRACT

An application of GluN2A and an NMDA receptor containing GluN2A as targets in screening antidepressant drugs, which may achieve rapid and low-cost screening of antidepressant drugs. Specifically, provided is a use of GluN2A, a binding fragment thereof or an NMDA receptor containing GluN2A in screening antidepressant drugs or in the preparation of a reagent for screening antidepressant drugs. Also provided are a method and apparatus for screening antidepressant drugs, and a kit.

### TECHNICAL FIELD

The present invention relates to the field of biopharmaceutical technology, specifically to the application of GluN2A and NMDA receptors containing GluN2A as targets in screening drugs for treating depression.

### BACKGROUND

Depression is an emotion related disease characterized by down in spirits and declining interest. With the accelerated pace of modern life and increasing social pressure, the incidence of depression is also increasing. As reported by World Health Organisation, there are about 300 million people suffering from depression worldwide. Patients suffer from long-term depression, delayed thinking, loss of interest in life, and in severe cases, loss of the ability for domesticity, study and work, even leading to suicidal tendencies and suicidal behaviours, which bring serious losses and burdens to the family and society. Therefore, the study of the pathogenesis of depression and the development of antidepressant drugs are particularly important.

Depression is a complex heterogeneous disease with causative factors originating from multiple aspects such as physiology, psychology, genetics, and society. In the past few decades, although a series of advances have been made in the field of neuroscience on the pathogenesis of depression, the exact pathogenesis has still not been elaborated in detail. Numerous studies and clinical trials have shown that the pathogenesis of depression involves multiple aspects, including decreased 5-HT and noradrenergic activity in the central nervous system, decreased BDNF expression, abnormally increased activity of the hypothalamicuitary-adrenal axis, increased inflammatory responses and decreased neuroregeneration and neuroplasticity.

The monoamine theory is the previously widely accepted possible mechanism for the pathogenesis of depression, that is reduced levels of monoamine neurotransmitters such as serotonin and norepinephrine in the brain lead to depression-related behaviours. The more commonly used first-line antidepressants in clinical practice are serotonin reuptake inhibitors (SSRI) and serotonin and norepinephrine reuptake inhibitors (SNRI). These two types of drugs can alleviate the symptoms of most depressed patients, but there are still some shortcomings, such as slow onset of effect and usually taking about one week to take effect. SSRI is not effective for nearly one-third of patients, and may even increase suicidal tendencies of the patients.

Therefore, there is an urgent need in this field to provide rapid and low-cost screening methods for antidepressants in order to provide more antidepressants.

### SUMMARY OF THE INVENTION

The purpose of the present invention is to provide a use of a GluN2A subtype NMDA receptor inhibitor as antidepressant drugs.

Another purpose of the present invention is to provide a fast and low-cost screening method and device for antidepressant drugs.

In a first aspect of the present invention, provided is a use of GluN2A, a binding fragment, or a NMDA receptor containing GluN2A in screening antidepressant drugs or in preparing a reagent for screening antidepressant drugs, wherein whether possessing the ability to inhibit and/or bind GluN2A, the binding fragment thereof, or the NMDA receptor containing GluN2A is used as a screening criterion for determining whether a drug candidate has antidepressant activity.

In another preferred embodiment, whether the drug candidate possessing the ability to increase the intrinsic excitability of excitatory neurons can further be used as a criterion for determining whether the drug candidate has antidepressant activity.

In another preferred embodiment, the screening is carried out in vitro; the method is non-diagnostic and non-therapeutic.

In another preferred embodiment, the GluN2A has an amino acid sequence of SEQ ID No: 1 (human GluN2A) or an amino acid sequence of at least 80% identity to SEQ ID No: 1, preferably, GluN2A has an amino acid sequence with at least 85%, at least 90%, or at least 95% identity to SEQ ID No: 1.

In another preferred embodiment, the GluN2A has an amino acid sequence of SEQ ID No: 2 (mouse GluN2A) or an amino acid sequence of at least 80% identity to SEQ ID No: 2, preferably, GluN2A has an amino acid sequence with at least 85%, at least 90%, or at least 95% identity to SEQ ID No: 2.

In another preferred embodiment, the NMDA receptor containing GluN2A is an NMDA receptor containing two GluN1s and two GluN2A subunits, or an NMDA receptor containing at least one GluN2A subunit.

In another preferred embodiment, the drug candidate is selected from the group consisting of: antibodies or binding fragments thereof, small molecule compounds or pharmaceutically acceptable salts thereof, and nucleic acids.

In another preferred embodiment, the depression includes phenotypes selected from the group consisting of: low spirits, delayed thinking, decreased volitional activity, suicidal tendency, cognitive impairment, somatic symptoms, or a combination thereof.

In a second aspect of the present invention, provided is a screening method for antidepressant drugs, comprising the steps of:
(i) providing GluN2A, a binding fragment thereof, and/or a NMDA receptor containing GluN2A;
(ii) contacting the drug candidate with GluN2A, the binding fragment thereof, and/or the NMDA receptor containing GluN2A; and
(iii) determining the inhibitory and/or binding ability of the drug candidate to GluN2A, the binding fragment, and/or the NMDA receptor containing GluN2A; when the drug candidate possesses the ability to inhibit and/or bind to GluN2A, the binding fragment thereof, and/or the NMDA receptor containing GluN2A, the drug candidate can be used as an antidepressant drug candidate.

In another preferred embodiment, the method further comprises a step of:
(iv) testing the effect of the drug candidate screened out in step (iii) on excitatory neurons; when the inducement of antidepressant like behavior and/or increase in the intrinsic excitability of excitatory neurons by the drug candidate is detected, the drug candidate can be used as antidepressant drugs.

In a third aspect of the present invention, provided is a screening device for antidepressant drugs, comprising a patch clamp device loaded with cells expressing GluN2A subtype NMDA receptors (such as HEK293, CHO, and Hela cells, etc.), wherein the device is configured to detect whether a candidate compound can reduce or block ion channel currents mediated by GluN2A subtype NMDA receptors.

In a fourth aspect of the present invention, provided is a screening device for antidepressant drugs, a suite for screening antidepressant drugs, comprising:
(a) a first patch clamp device, wherein the first patch clamp device is a patch clamp device loaded with cells expressing GluN2A subtype NMDA receptors (such as HEK293, CHO, and Hela cells, etc.); and
(b) a second patch clamp device, wherein the second patch clamp device is a patch clamp device loaded with tissue sections of hippocampal from animals (such as rats, mice, etc.) or primary cultured neurons, wherein the device is configured to detect whether the candidate compound can enhance the intrinsic excitability of excitatory neurons.

In a fifth aspect of the present invention, provided is a screening kit for antidepressant drugs, comprising:
GluN2A, the binding fragment thereof, and/or the NMDA receptor containing GluN2A, which act as a reagent for screening antidepressants.

In another preferred embodiment, GluN2A, its binding fragments, and/or NMDA receptors containing GluN2A are used as targets for screening antidepressant drugs.

In a sixth aspect of the present invention, provided is a use of a GluN2A subtype NMDA receptor inhibitor in the preparation of pharmaceutical compositions for antidepressant drugs.

In another preferred embodiment, the GluN2A subtype NMDA receptor inhibitor is selected from the group consisting of: an antibody or a binding fragment thereof, a small molecule compound or a pharmaceutically acceptable salt thereof, and a nucleic acid.

In another preferred embodiment, the GluN2A subtype NMDA receptor inhibitor is a compound selected from the group consisting of:

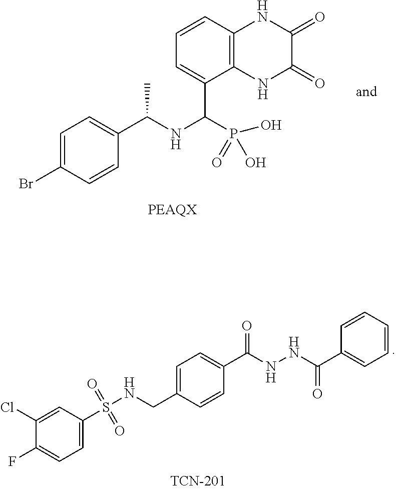 [SMILES: C[C@H](NC(c1cccc2[nH]c(=O)c(=O)[nH]c12)P(=O)(O)O)c1ccc(Br)cc1 ; O=C(NNC(=O)c1ccc(CNS(=O)(=O)c2ccc(F)c(Cl)c2)cc1)c1ccccc1]

or a pharmaceutically acceptable salt thereof.

In another preferred embodiment, the GluN2A subtype NMDA receptor inhibitor is not MK-801 or ketamine.

In another preferred embodiment, the GluN2A subtype NMDA receptor inhibitor is a monoclonal or polyclonal antibody thereof.

In another preferred embodiment, the pharmaceutical composition further includes pharmaceutically acceptable carriers.

In a seventh aspect of the present invention, provided is a method for treating depression, comprising a step of: administering an effective amount of GluN2A subtype NMDA receptor inhibitor to a subject suffering from depression, thereby treating depression.

In another preferred embodiment, the subject is a mammal, such as human, rats and mice.

It should be understood that, within the scope of the present invention, each of the above technical features of the present invention and each of the technical features specifically described in the following (such as the embodiments) can be combined with each other to constitute a new or preferred technical solution. Due to space limitations, It will not be repeated herein.

### DESCRIPTION OF THE DRAWINGS

FIG. 1 shows the GluN2A protein expression content in wild-type (WT) mice, GluN2A knockout mice and conditional knockout mice.

FIG. 2 shows the behavioral experimental results of wild-type (WT) mice and GluN2A knockout mice. (A) Open field experiment; (B) forced swim test and tail-suspension experiment.

FIG. 3 shows the correlation analysis between immobility time in forced swim test and activity distance in open field experiment of wild-type (WT) mice and GluN2A knockout mice.

FIG. 4 shows the immobility time of wild-type (WT) mice and GluN2A knockout mice in a forced swim test after treatment with NMDA receptor antagonists. (A) MK-801; (B) Ketamine.

FIG. 5 shows the immobility time in forced swimming after administration of MK-801 to GluN2A conditional knockout mice.

FIG. 6 shows the immobility time of wild-type (WT) mice and GluN2A knockout mice in forced swim test and tail suspension experiment after treatment with the GluN2B selective inhibitor Ro 25-6981. (A) forced swim test; (B) tail-suspension test.

FIG. 7 shows that knocking out the GluN2A subtype NMDA receptor after being an adult can alleviate depressive-like behavior in mice. (A) Experimental process diagram; (B, C) The number of avoidance failures and avoidance latency of Test 1; (D, E) The number of avoidance failures and avoidance latency of Test 2; (F) The immobility time of mice in tail suspension experiment after knocking out the GluN2A subtype NMDA receptor in adulthood.

FIG. 8 shows that the knockdown of GluN2A subtype NMDA receptors in mice increases the intrinsic excitability of excitatory neurons in the hippocampus. (A) Intrinsic excitability of excitatory neurons in the hippocampus, when knocking out the GluN2A subtype NMDA receptor during the developmental stage. (B) Intrinsic excitability of excitatory neurons in the hippocampus, after knocking out the GluN2A subtype NMDA receptor in adulthood.

FIG. 9 shows that the knockdown of GluN2A subtype NMDA receptor eliminates the increased intrinsic excitability of excitatory neurons in hippocampus induced by NMDA receptor antagonist MK-801 and ketamine (Ket). (A) Intrinsic excitability of excitatory neurons in the hippocampus of WT mice treated with MK-801. (B) Intrinsic excitability of excitatory neurons in the hippocampus of GluN2A knockout mice treated with MK-801. (C) Intrinsic excitability of excitatory neurons in the hippocampus of WT mice treated with ketamine. (D) Intrinsic excitability of excitatory neurons in the hippocampus of GluN2A knockout mice treated with ketamine.

FIG. 10 shows that the selective inhibition of GluN2B does not affect the increase in intrinsic excitability of hippocampal excitatory neurons induced by MK-801. (A) Intrinsic excitability of excitatory neurons in the hippocampus of WT mice treated with GluN2B selective inhibitor Ro 25-6981. (B) Intrinsic excitability of excitatory neurons in the hippocampus of WT mice treated with MK-801 and Ro 25-6981.

FIG. 11 shows that the specific blockade of Ro 25-6981 on GluN2B does not mask the rapid antidepressant like behavior induced by MK-801 in WT mice. (A) The immobility time of WT mice in tail suspension tests; (B) The immobility time of WT mice in forced swim tests.

### EMBODIMENTS FOR CARRYING OUT THE INVENTION

After extensive and in-depth research and through extensive screening and testing, the present inventors provide the use of GluN2A and GluN2A-containing NMDA receptors as targets in the screening of antidepressant drugs. The present invention discovered for the first time that induced knockout of GluN2A alleviates depressive-like behaviors of mice in the mouse model of adult learned helplessness depression, indicating that inhibiting and/or eliminating the function of GluN2A can be used to treat depressive symptoms in patients. In addition, the knockout of GluN2A eliminates the rapid antidepressant effects of NMDA receptor subtype non selective antagonists MK-801 and ketamine, while fails to block this effect of MK-801 when GluN2B being inhibited, indicating that the rapid antidepressant effects of such drugs require the presence of GluN2A rather than GluN2B. The above results demonstrate that GluN2A can serve as a target for developing new antidepressant drugs. In addition, the antidepressant effect caused by knocking out GluN2A (rather than inhibiting GluN2B) is associated with an increase in intrinsic excitability of excitatory neurons, suggesting that enhancing the intrinsic excitability of excitatory neurons can also serve as a criterion for screening antidepressant drugs. The present invention is completed on this basis.

### Terms

Unless otherwise defined, all technical and scientific terms used herein have the same meaning as would normally be understood by those of ordinary skill in the art to which the invention belongs.

As used herein, term “about” means that the value may change by no more than 1% from the enumerated value when used in reference to a specific enumerated value. For example, as used herein, the expression “about 100” includes all values between 99 and 101 and (e. g., 99.1, 99.2, 99.3, 99.4, etc.).

As used herein, term “contain” or “include (comprise)” may be open, semi-closed, and closed. In other words, said term also includes “consisting essentially of” or “consisting of”.

As used herein, the term “room temperature” or “ordinary temperature” refers to a temperature of 4-40° C., preferably 25±5° C.

### NMDA Receptor

NMDA receptor refers to N-methyl-D-aspartate receptor, which is a subtype of ionotropic glutamate receptors. NMDA is a tetramer composed of two essential GluN1 and two variable GluN2 or GluN3 subunits. Abnormal NMDA receptor function is associated with various neurodegenerative diseases such as Alzheimer's disease, Parkinson's syndrome, Huntington's chorea, as well as psychiatric disorders such as autism, schizophrenia, and depression.

### GluN2A, and NMDA Receptors Containing GluN2A

GluN2 contains four subtypes: GluN2A, GluN2B, GluN2C, and GluN2D. Structurally, each subtype contains four structural domains: Extracellular domain (N-terminal domain), Agonist binding domain, Transmembrane domain, and intracellular domain (C-terminal domain). However, its expression varies in different brain regions and changes according to different developmental stages. The diversity of NMDA receptor subunits thus increases the complexity of studying the pathogenesis of depression.

Among them, GluN2A of the present invention can be of various groups of mammals, such as primates, rats, etc., preferably human, rats, and mice.

Preferably, the GluN2A has an amino acid sequence of SEQ ID No: 1 (human GluN2A) or an amino acid sequence of at least 80% identity to it, preferably, GluN2A has an amino acid sequence of at least 85%, at least 90%, or at least 95% homology identity to SEQ ID No: 1.

Preferably, the GluN2A has an amino acid sequence of SEQ ID No: 2 (mouse GluN2A) or an amino acid sequence of at least 80% identity to it, preferably, GluN2A has an amino acid sequence of at least 85%, at least 90%, or at least 95% identity to SEQ ID No: 2.

SEQ ID No: 1
mgrvgywtll vlpallvwrg papsaaaekg ppalniavml ghshdvtere lrtlwgpeqa
aglpldvnvv allmnrtdpk slithvcdlm sgarihglvf gddtdqeava qmldfissht
fvpilgihgg asmimadkdp tstffqfgas iqqqatvmlk imqdydwhvf slvttifpgy
refisfvktt vdnsfvgwdm qnvitldtsf edaktqvqlk kihssvilly cskdeavlil
searslgltg ydffwivpsl vsgntelipk efpsglisvs yddwdyslea rvrdgigilt
taassmlekf syipeakasc ygqmerpevp mhtlhpfmvn vtwdgkdlsf teegyqvhpr
lvvivlnkdr ewekvgkwen htlslrhavw pryksfsdce pddnhlsivt leeapfvive
didpltetcv rntvpcrkfv kinnstnegm nvkkcckgfc idilkklsrt vkftydlylv
tngkhgkkvn nvwngmigev vyqravmavg sltineerse vvdfsvpfve tgisvmvsrs
ngtvspsafl epfsasvwvm mfvmllivsa iavfvfeyfs pvgynrnlak gkaphgpsft
igkaiwllwg lvfnnsvpvq npkgttskim vsvwaffavi flasytanla afmiqeefvd
qvtglsdkkf qrphdysppf rfgtvpngst ernirnnypy mhqymtkfnq kgvedalvsl
ktgkldafiy daavlnykag rdegcklvti gsgyifattg ygialqkgsp wkrqidlall
qfvgdgemee letlwltgic hneknevmss qldidnmagv fymlaaamal slitfiwehl
fywklrfcft gvcsdrpgll fsisrgiysc ihgvhieekk kspdfnltgs qsnmlkllrs
aknissmsnm nssrmdspkr aadfiqrgsl imdmvsdkgn lmysdnrsfq gkesifgdnm
nelqtfvanr qkdnInnyvf qgqhpltlne snpntvevav steskansrp rqlwkksvds
irqdslsqnp vsqrdeatae nrthslkspr ylpeemahsd isetsnratc hrepdnsknh
ktkdnfkrsv askypkdcse vertylktks ssprdkiyti dgekepgfhl dppqfvenvt
lpenvdfpdp yqdpsenfrk gdstlpmnrn plhneeglsn ndqyklyskh ftlkdkgsph
setseryrqn sthersclsn mptysghftm rspfkcdacl rmgnlydide dqmlqetgnp
atgeqvyqqd waqnnalqlq knklrisrqh sydnivdkpr eldlsrpsrs islkdrerll
egnfygslfs vpssklsgkk sslfpqgled skrsksllpd htsdnpflhs hrddqrlvig
rcpsdpykhs lpsqavndsy lrsslrstas ycsrdsrghn dvyisehvmp yaanknnmys
tprvlnscsn rrvykkmpsi esdv
SEQ ID No: 2
mgrlgywtll vlpallvwhg paqnaaaekg tpalniavll ghshdvtere Irnlwgpeqa
tglpldvnvv allmnrtdpk slithvcdlm sgarihglvf gddtdqeava qmldfissqt
fipilgihgg asmimadkdp tstffqfgas iqqqatvmlk imqdydwhvf slvttifpgy
rdfisfiktt vdnsfvgwdm qnvitldtsf edaktqvqlk kihssvilly cskdeavlil
searslgltg ydffwivpsl vsgntelipk efpsglisvs yddwdyslea rvrdglgilt
taassmlekf syipeakasc ygqtekpetp lhtlhqfmvn vtwdgkdlsf teegyqvhpr
lvvivlnkdr ewekvgkwen qtlslrhavw pryksfsdce pddnhlsivt leeapfvive
didpltetcv rntvporkfv kinnstnegm nvkkcckgfc idilkklsrt vkftydlylv
tngkhgkkvn nvwngmigev vyqravmavg sltineerse vvdfsvpfve tgisvmvsrs
ngtvspsafl epfsasvwvm mfvmllivsa iavfvfeyfs pvgynrnlak gkaphgpsft
igkaiwllwg lvfnnsvpvq npkgttskim vsvwaffavi flasytanla afmiqeefvd
qvtglsdkkf qrphdysppf rfgtvpngst ernirnnypy mhqymtkfnq rgvedalvsl
ktgkldafiy daavlnykag rdegcklvti gsgyifattg ygialqkgsp wkrqidlall
qfvgdgemee letlwltgic hneknevmss qldidnmagv fymlaaamal slitfiwehl
fywklrfcft gvcsdrpgll fsisrgiysc ihgvhieekk kspdfnltgs qsnmlkllrs
aknisnmsnm nssrmdspkr aadfiqrgsl ivdmvsdkgn liysdnrsfq gkdsifgenm
nelqtfvanr hkdslsnyvf qgqhpltlne snpntvevav steskgnsrp rqlwkksmes
Irqdslnqnp vsqrdektae nrthslkspr ylpeevahsd isetssratc hrepdnnknh
ktkdnfkrsm askypkdcse vertyvktka ssprdkiyti dgekepsfhl dppqfieniv
lpenvdfpdt yqdhnenfrk gdstlpmnrn plhnedglpn ndqyklyakh ftlkdkgsph
segsdryrqn sthersclsn lptysghftm rspfkcdacl rmgnlydide dqmlqetgnp
atreeayqqd wsqnnalqfq knklkinrqh sydnildkpr eidlsrpsrs islkdrerll
egnlygslfs vpsskllgnk sslfpqgled skrsksllpd htsdnpflht ygddqrlvig
rcpsdpykhs lpsqavndsy lrsslrstas ycsrdsrghs dvyisehvmp yaanknnmys
tprvlnscsn rrvykkmpsi esdv

### Depression

Depression is the most common major depressive disorder with significant low mood as the main clinical feature, and is a typical type of mood disorder. The clinical manifestations of depression include: low spirits, thinking slow, bulesis activity decline, cognitive impairment (such as memory decay, attention disorders, etc.), and physical symptoms (such as sleep disorders, fatigue, vomiting, palpitations, etc.).

In the present invention, the depression includes depressive symptoms of Major Depressive Disorder, as well as depressive symptoms in other diseases such as manic depression, schizophrenia, etc.

As used herein, the terms “antidepressant”, “antidepression”, or “treating depression” can be used interchangeably to refer to partial or complete relief, reversal, or treatment of depression. In particular, the antidepressant does not include preventing the formation of depression. In particular, the depression is not caused by genetic factors or the genetic factor is not the primary cause of the depression. In particular, the subject is a depressed patient with normal brain development.

### Antidepressant Drug Screening Methods

The inventor unexpectedly discovers that knocking out or inhibiting GluN2A can reverse/treat depression. In addition, the existing antidepressant drugs require the presence of GluN2A to demonstrate antidepressant effects.

Therefore, the present invention provides a use of GluN2A, its binding fragments or NMDA receptors containing GluN2A in screening antidepressant drugs or in the preparation of a reagent for screening antidepressant drugs. In the present invention, the ability to inhibit and/or bind to GluN2A, its binding fragments, or NMDA receptors containing GluN2A can be used as a screening criterion for determining whether a drug candidate has antidepressant activity.

Furthermore, the present invention also provides a screening method for antidepressant drugs, comprising the steps of:
(i) providing GluN2A, its binding fragments, and/or NMDA receptors containing GluN2A;
(ii) contacting the drug candidate with GluN2A, its binding fragments, and/or NMDA receptors containing GluN2A; and
(iii) determining the inhibitory and/or binding ability of the drug candidate to GluN2A, its binding fragments, and/or NMDA receptors containing GluN2A; when the drug candidate has the ability to inhibit and/or bind to GluN2A, its binding fragments, and/or NMDA receptors containing GluN2A, the drug candidate can be used as an antidepressant drug candidate.

In another preferred embodiment, the method further comprises steps of:
(iv) testing the effect of the drug candidate screened out in step (iii) on xcitatory neurons; when the inducement of antidepressant like behavior and/or increase of the intrinsic intrinsic excitability of excitatory neurons resulted from the drug candidate, the drug candidate can be used as an antidepressant drug.

The screening method of the present invention may be performed at the molecular, cellular, tissue or animal level. Preferably, the method is carried out in vitro.

As used herein, “NMDA receptor containing GluN2A”, “GluN2A subtype NMDA receptor” and “NMDA receptor containing GluN2A subunit” used for screening antidepressant drugs can be used interchangeably, referring to a NMDA receptor containing at least one GluN2A subunit, such as a NMDA receptor containing one or two GluN2A subunits; preferably the receptor includes two GluN2A subunits.

Those skilled in this field understand that the inhibitory and/or binding ability of the drug candidate to GluN2A, its binding fragments, and/or the NMDA receptor containing GluN2A can be determined according to methods commonly used in the art, including but not limited to molecular experiments, cell experiments, and/or animal experiments. Typical detection methods include, but are not limited to: patch clamp, western blot assay, high-performance liquid chromatography, electrophoresis, mass spectrometry, immunofluorescence, or a combination thereof. To determine the IC₅₀ values of drug candidates for inhibiting GluN2A, its binding fragments, and/or NMDA receptor containing the same, it can usually be compared to positive or negative control drugs.

As used herein, the terms “inhibition”, “inhibitory ability”, “decrease” or “blockade” refer to the inhibitory effect on the function of GluN2A subtype NMDA receptors, such as the inhibitory effect on sodium, potassium, calcium signals (currents) mediated by GluN2A subtype NMDA receptors or other downstream signaling pathways or effects coupled with them (such as phosphorylation of Erk or intrinsic excitability of excitatory neurons). Preferably, the administration of the drug candidate or inhibitor of the present invention results in a decrease of at least 10% (preferably, at least 20%, at least 40%, at least 50%, at least 80%, or at least 100%) in the function of the GluN2A subtype NMDA receptor.

In another preferred embodiment, drugs can be screened by detecting the binding ability of the drug candidate to GluN2A, its binding fragments, and/or NMDA receptors containing GluN2A. For example, compared to the binding ability C0 of substrates or positive control drugs to GluN2A, its binding fragments, and/or NMDA receptors containing GluN2A, the binding ability CI of the drug candidates or inhibitors of the present invention to GluN2A, its binding fragments, and/or NMDA receptors containing GluN2A is at least 10% of C0, preferably, is at least 20%, at least 40%, at least 50%, at least 80%, or at least 100%, or even 200% or 300% or more of C0.

### Drug Screening Device or Kit

The present invention provides a patch clamp device for screening antidepressant drugs.

In the present invention, the patch clamp device for screening antidepressant drugs screens antidepressant drugs by detecting whether a drug candidate can block the ion channel current mediated by NMDA receptors containing GluN2A on the cell membrane. That is to say, in a preferred embodiment, the patch region is the region where the NMDA receptor containing GluN2A is located.

The patch clamp technology is a microelectrode technique that records the electrical activity of ion channels on biofilms by clamping voltage or current. The patch clamp technology is widely used in this field and its device structure is known to those skilled in the art. Typically, the patch clamp device may include components selected from the group consisting of: mechanical components (shock-proof workbench, shielding cover, equipment rack), optical components (microscope, video monitor, monochromatic light system), electronic components (patch clamp amplifier, stimulator, data acquisition equipment, computer system), and micromanipulators. Alternatively, commercially available patch clamp devices such as MultiClamp or HEKA systems can be used.

Preferably, the cells that can be used in the patch clamp device include: cells overexpressing NMDA receptors comprising human or mouse GluN2A subtype, such as (but not limited to) HEK293, CHO, or HELA cells, or African Xenopus oocytes, etc. Preferably, the cells are HEK293 cells overexpressing NMDA receptors comprising human or mouse GluN2A subtype. Usually, during drug screening, each drug candidate can be tested using three or more cells.

Preferably, the present invention also provides a suite for screening antidepressant drugs, comprising:
(a) a first patch clamp device loaded with cells expressing GluN2A subtype NMDA receptors, and the device is configured to detect whether a candidate compound can reduce or block ion channel currents mediated by GluN2A subtype NMDA receptors (through such as whole cell recording of patch clamp device); and
(b) a second patch clamp device, wherein the device is a patch clamp device loaded with tissue sections of hippocampus from animal brain or primary cultured neurons (such as rats, mice, etc.), wherein the device is configured to detect whether the candidate compound can enhance the intrinsic excitability of excitatory neurons.

Usually, the suite also includes instructions etc.

The suite can perform rapid initial screening through the first patch clamp device, and then re-screen the drug candidate that pass the initial screening through the second patch clamp device, achieving rapid and highly accurate screening of antidepressant drugs.

### a Screening Kit for Antidepressant Drugs

The screening kit for antidepressant drugs of the present invention includes GluN2A, its binding fragments, and/or NMDA receptors containing GluN2A, as reagents for screening antidepressant drugs.

Typically, the kit also includes containers, instructions, etc.

### GluN2A Inhibitors, Pharmaceutical Compositions and Uses Therefor

The present invention provides a use of GluN2A subtype NMDA receptor inhibitors in the preparation of pharmaceutical compositions for antidepressant drugs.

In another preferred embodiment, the GluN2A subtype NMDA receptor inhibitor is selected from the group consisting of: antibodies or binding fragments thereof, small molecule compounds or pharmaceutically acceptable salts thereof, and nucleic acids.

In another preferred embodiment, the GluN2A subtype NMDA receptor inhibitor is a substance known to have inhibitory activity against GluN2A, or a substance screened out through the drug screening methods of the present invention that has inhibitory and/or binding ability against GluN2A, its binding fragments, and/or NMDA receptors containing it.

In the present invention, the pharmaceutical composition comprises GluN2A subtype NMDA receptor inhibitors as antidepressant active ingredients and pharmaceutically acceptable carriers.

“Pharmaceutically acceptable excipients” and “pharmaceutically acceptable carriers” refer to substances that facilitate the formulation and/or administration and/or absorption by individuals of active agents, and can be included in the composition disclosed herein without causing significant adverse toxicological effects on the individual. Non limiting examples of pharmaceutically acceptable carriers and excipients include water, NaCl, saline solution, lactated Ringer's solution, conventional sucrose, conventional glucose, binders, fillers, disintegrants, lubricants, coatings, sweeteners, flavor agents, salt solutions (such as Ringer's solution), alcohols, oils, gelatin, carbohydrates (such as lactose, linear starch or amylose), fatty acid esters, hydroxymethylcellulose, polyvinylpyrrolidine and pigments, etc. Such formulations can be sterilized and, if necessary, mixed with auxiliary agents such as lubricants, preservatives, stabilizers, wetting agents, emulsifiers, salts that affect osmotic pressure, buffering agents, colorants, and/or aromatic substances that will not react harmfully with or interfere with the activity of the compounds provided herein. Ordinary technical personnel in this field will recognize that other drug carriers and excipients are suitable for use with inhibitors of the present invention.

In certain embodiments, the pharmaceutical composition of the present invention may be in solid or liquid form.

The active ingredient can be administered to the subject through any suitable route, including oral administration, parenteral administration, inhalation of spray, topical administration, rectal administration, nasal administration, sublingual administration, vaginal administration or via an implantable kit. The term “parenteral” as used herein includes subcutaneous, intravenous, intramuscular, intra-articular, synovial, intrasternal, intrathecal, intrahepatic, intralesional, and intracranial injection or infusion techniques. Preferably, the composition is administered orally, intraperitoneally or intravenously.

The pharmaceutical composition of the present invention suitable for oral administration will typically be in the form of solid discrete units, such as tablets, capsules, cachets, powders, granules, lozenges, patches, suppositories, pills, or in liquid form, such as liquid formulations, injectable or infusible solutions or suspensions.

The precise amount of active ingredients that provide therapeutic efficacy to an individual will depend on the mode of administration, the type and severity of the disease and/or condition, as well as characteristics of the individual, such as general health status, age, gender, weight, and drug tolerance. Those skilled in the art will be able to determine the appropriate dosage based on these and other factors. When co-administered with other therapeutic agents, the “effective therapeutic amount” of any other therapeutic agent will depend on the type of medication used. The appropriate dosage for approved therapeutic agents is known and can be adjusted by those skilled in the art based on the individual's condition, the type of disease being treated, and the amount of compounds of the present invention used based on the following, such as the dosage reported in the literature and recommended in the Physician's Desk Reference (57th edition, 2003). Preferably, the composition should be formulated in such a way that an inhibitor dose of 0.01-100 mg/kg body weight/day can be administered to patients receiving these compositions. In certain embodiments, the composition of the present invention provides doses ranging from 0.01 mg to 50 mg. In other embodiments, doses of 0.1 mg-25 mg or 5 mg-40 mg are provided.

The present invention also provides a method for treating depression, comprising a step of: administering an effective amount of GluN2A subtype NMDA receptor inhibitors to a subject suffering from depression, thereby treating depression.

Examples of subjects for administration with the pharmaceutical compositions or therapeutic agents of the present invention include mammals (e.g., humans, mice, rats, hamsters, rabbits, cats, dogs, cows, sheep, monkeys, etc.). In another preferred embodiment, the subject is an individual with fully developed brain, such as a subject who only suffers from depression in adulthood. For humans, the age of the subject for administration is ≥16 years old, ≥18 years old, or ≥20 years old.

The main advantages of the present invention include:

1. The present invention demonstrates for the first time that inhibiting GluN2A can be used to reverse/treat depression, thus making it possible to develop specific drugs for treating depression.

2. The present invention provides the use of GluN2A inhibitors in the treatment of depression. Compared with general NMDA receptor inhibitors, the use of specific GluN2A inhibitors can reduce drug side effects.

3. The present invention also provides a screening method, screening device, or screening kit for screening antidepressant drugs. By taking GluN2A as a drug target, rapid and low-cost high-throughput screening of antidepressant drugs can be achieved.

The present invention was further described hereafter in combination with specific examples. It should be understood that these examples are only used to illustrate the and not to limit the scope of the invention. The experimental methods with no specific conditions described in the following examples are generally performed under the conventional conditions, for example, according to J. Sambrook et al., Molecular Cloning: A Laboratory Manual, 3rd Edition, Science Press, 1989, or according to the manufacture's instructions. Unless otherwise stated, percentages and parts are by weight.

### Experiment Animals

GluN2A complete gene knockout mice were purchased from the Japanese Institute of Physical and Chemical Research (RIKEN) and bred at Shanghai Model Organisms Center, Inc., resulting in GluN2A wild-type (WT) and GluN2A knockout mice (KO). Both of Grin2a^{flox/flox} mice and UBC-CreERT mice were purchased from Shanghai Model Organisms Center, Inc. These two types of mice were bred and mated to obtain UBC CreERT/Grin2a^{flox/flox} mice. After UBC-CreERT/Grin2a^{flox/flox} mice being administrated with tamoxifen, Cre recombinase expression were induced, thereby obtaining GluN2A conditional knockout mice. Mice were incubated under standard conditions with a humidity of 50±10% and a temperature of 22-25° C., with a 12 hour to 12 hour circadian rhythm (nighttime: 8 p.m. to 8 a.m.), fed with standard food and water. All experiments were conducted in accordance with the recommendations of the Code of Ethics for the Management and Use of Experimental Animals in Universities of the Chinese Academy of Sciences.

Drug: MK-801 (behavioral experiment: 10 mg/kg; electrophysiology: 10 M), ketamine (behavioral experiment: 10 mg/kg; electrophysiology: 20 M), tamoxifen (2 mg/mouse), Ro 25-6981 (behavioral experiment: 10 mg/kg; electrophysiology: 3 M).

Antibody: GluN2A antibody was purchased from Novusbio USA; α/β-Tubulin antibodies were purchased from Cell Signaling Technology USA.

Reagents: Tetrodotoxin was purchased from Shanghai Wokai Biotechnology Co., Ltd., Picrotoxin was purchased from Sigma Corporation USA, and NBQX and Ro 25-6981 were purchased from TOCRIS Corporation USA.

Choline chloride solution: CholineCl 110 mM, NaHCO₃25 mM, MgSO₄·7H₂O 7 mM, KCl 1.25 mM, NaH₂PO₄·2H₂O 1.25 mM, CaCl₂) 0.5 mM, D-Glucose 25 mM, Na ascorbate 11.6 mM, Na Pyruvate 3.1 mM.

Artificial cerebrospinal fluid (ACSF): NaCl 127 mM, KCl 2.5 mM, NaH₂PO₄·2H₂O 1.25 mM, NaHCO₃25 mM, CaCl₂) 2.5 mM, MgCl₂ 1.3 mM, D-Glucose 25 mM.

Electrode internal fluid: 120 mM K-gluconate, 20 mM KCl, 10 mM HEPES, 10 mM Na phosphocreatine, 4 mM Na₂ATP, 0.3 mM Na₃GTP, 2.5 mM MgCl₂ and 0.5 mM EGTA (pH 7.25).

### EXAMPLE

### I. Experimental Method

### 1. Open Field Test:

Open Field Test (OFT) box (with soundproof box, made of opaque organic glass, inner diameter 40*40*40 cm); analysis software was purchased from Nodus (Ethovision XT 11.5). The experiment was conducted in a quiet environment, and the animals were weighed and acclimated to the experimental room for at least 30 minutes before the experiment. Before the experiment, the software Etho Vision XT 11.5 was set up, and mice aged 12 weeks and above were placed in the center of the bottom surface of the box (40 cm*40 cm), and the activity of the mice was recorded for 1 hour. After each experiment, 75% alcohol was used to wipe the instrument to avoid information left from the previous round of animals (for example, odors of such as animals' stool and urine) from affecting the results of the next test. Replace with the next round of animal and continue the experiment. Data were collected at the end of the experiment for statistical analysis.

### 2. Forced-Swim Test:

The mice were placed in a transparent organic glass water tank with a diameter of 25 cm and a height of 30 cm, which was filled with 15 cm high of water with a temperature of 23-25° C., and the total recording time was 6 min. The immobility time of the mice within the latter 4 min was analysed, reflecting the degree of helplessness and despair of the mice.

### 3. Tail Suspension Test:

After fixing the mice at about 1.5 cm from the distal end of the tail, the mice were suspended with their heads facing downwards at a distance of about 35 cm from the ground. The total duration of recording was 6 min, and the percentage of time that the mice remained stationary during the latter 4 min was analysed to reflect the degree of desperation of mice.

### 4. Learned Helplessness Test:

Adult mice were placed in a shuttle box (Panlab, Barcelona, Span) containing two equally sized compartments for conducting inescapable electrical stimulation and learned helplessness experiments. This device can provide both conditioned stimulation (CS) (i.e. sound and light) and unconditioned stimulation (US) (i.e. footshock to mice through the compartment floor), controlled by Shuttavoid software (Panlab, Barcelona, Spain). Firstly, the mice were subjected to three consecutive days of training experiments: the mice were placed in any compartment to adapt to the environment for 3 minutes, and then the door between the two compartments were closed. 50 unpredictable footshocks (0.2 mA, 5 seconds) were given at a random interval of 15-35 seconds. The control group mice were not subjected to electric shock during the training stage, and the rest of the conditions were the same. In the stage of learned helplessness test, the conditions were the same as in the training stage except that the door between the two compartments was kept open and the electric shock was terminated after the mice escaped to the other compartment. The learned helplessness phenotype of mice was assessed using escape failure rate (%) and escape latency(s).

### 5. Acute Administration of MK-801, Ketamine, and Ro25-6981:

MK-801 at a dose of 0.1 mg/kg, ketamine at a dose of 10 mg/kg, and Ro 25-6981 at a dose of 10 mg/kg were administrated to mice by intraperitoneal injection. Behavioural experiments such as subsequent open field tests, tail suspension tests, and forced swim tests were carried out 1 hour post intraperitoneal injection.

### 6. Western Blotting Assay:

An appropriate amount of brain tissue was taken into 1.5 ml EP tube, the EP tube was added with 0.5-1 ml RIPA lysis solution, then added with one bead for each EP tube, lysed ifor 4-6 min in Tissue Lyser, then centrifugated at 12,000 rpm for 10 min. The protein concentration in the supernatant was measured using the Prierce TM BCA protein assay kit (purchased from Thermo Scientific, USA), and then 8 g of protein was taken for Western blotting analysis. The protein samples were separated by sodium dodecyl sulfate polyacrylamide gel electrophoresis (SDS-PAGE) and transferred to PVDF membrane. The membranes were placed in 1% BSA-TBST blocking solution at room temperature for 1-2 h, then incubated with a 1:1000 diluted primary antibody at 4° C. overnight, followed by incubation with a 1:1000 diluted secondary antibody at room temperature for 1-2 h.

### 7. Preparation of Brain Slices and Electrophysiological Recording:

### Preparation of Brain Slices:

The beaker containing choline chloride slice solution was placed in an ice box and continuously introduced with a mixture of 95% O₂ and 5% CO₂ to saturate it with oxygen for use. After anesthetized with isoflurane, mice aged 4-8 weeks were quickly decapitated and euthanized. After exposing the skull and peeling off the dura mater, the whole brain was placed in a pre-cooled choline chloride solution for about 1 minute to reduce brain temperature. The whole brain was then taken out and the bilateral hippocampus were peeled off and attached to the surface of the agar block with one end of the hippocampus close to one of the edges of the agar block, after which the hippocampus-attached agar block was glued with 502 adhesive to a removable tray of the oscillating slicer, which was secured in the slicing tank of the oscillating slicer filled with pre-cooled choline chloride slicing solution. The oscillating slicer blade was adjusted to the horizontal position, then start the slicer to begin slice. The speed of the oscillating slicer was set to about 0.2 mm/s, and the slice thickness was 400 μm. The sliced hippocampus was carefully placed into ACSF preheated in water bath at 33-34° C. using a rubber hose and incubated for 30-60 minutes with a continuous introduction of a gas mixture of 95% O₂ and 5% CO₂. After incubation at 33-34° C., the vessel containing the brain slices was removed from the water bath and incubated at room temperature for about 30 min with continuous aeration and then electrophysiological recordings were performed. Hippocampal slices can be used for about 6 hours under room temperature with continuous aeration.

### 8. Whole Cell Recording:

Hippocampal slices after incubation were carefully transferred to the bath of the microscope carrier stage with a pipette, and were held in place by pressing using an anchor attached to parallel nylon filaments, using a peristaltic pump to keep the bath continuously pumped with oxygen-saturated ACSF, and the temperature was maintained at room temperature of about 23-26° C. The CA1 region of the hippocampus was firstly localised at low magnification (10×), then switching to high magnification (40×) to locate CA1, after which the field of view was switched to the monitor to look for pyramidal neurons with good condition in hippocampal CA1. Neurons with good condition exhibit strong cellular three-dimensional sense, clear contour, no swelling, no nucleolus appearance, elastic cell membrane, and “dimple-like” depression after electrode contact. In the experiment, a P-1000 electrode drawing instrument was used to draw glass electrodes using a three-step method, making the impedance of the electrode into the liquid 4-6 MS2. Prior to electrode into the liquid, a 10 ml syringe was used to apply about 2 ml or so of positive pressure into the electrode to prevent clogging of the electrode tip, and the baseline of the recording software was reset to zero using the Multiple 700B. A microelectrode operator was applied to slowly approach the electrode to the surface of the cell. When the electrode touched the surface of the cell, a “dimple-shaped” depression would appear. At this time, positive pressure would be immediately released and appropriate negative pressure would be applied to form a seal between the electrode and the cell. The change of sealing resistance was observed through the recording software, and when the impedance reached GΩ, it indicated that a high-resistance seal was formed between the cell and the electrode. After stabilizing for about 30-60 seconds, negative pressure was applied to break the membrane, and at this moment, a whole cell mode was formed. In current clamp mode, a depolarized step current stimulation of 10-330 pA was applied, with a step current of 20 pA. The intrinsic excitability of the neuron was evaluated by analyzing the number of action potentials generated by the neuron.

### 9. Data Analysis:

All values are expressed as mean±standard error (SEM). Statistical analysis was performed using the t-test and p<0.05 was considered statistically significant.

### II. Experimental Results

### 1. Identification of GluN2A Complete Gene Knockout Mice and Conditional Gene Knockout Mice

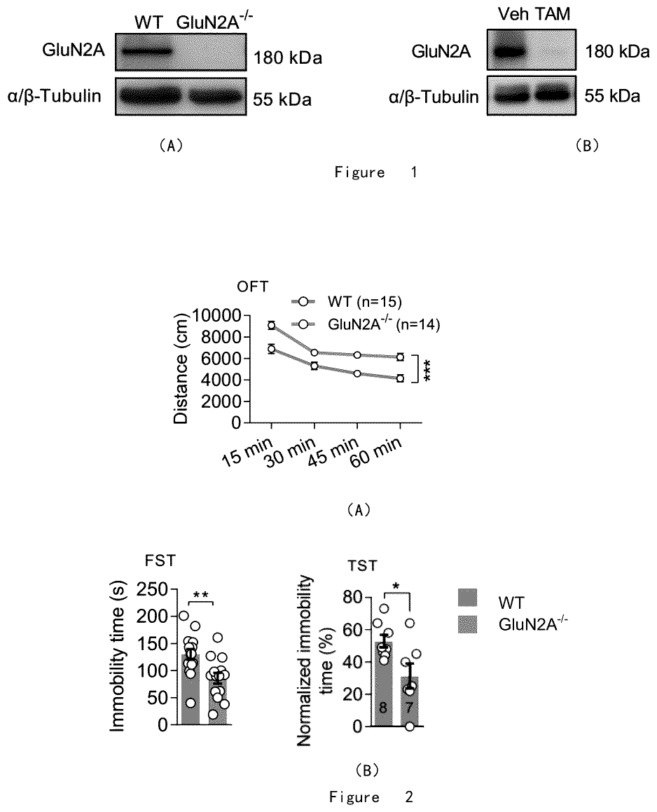

Mouse hippocampal tissue was taken for western blotting analysis. As shown in FIG. 1A, compared with WT mice, the expression of GluN2A protein in KO mice was significantly reduced, indicating the successful construction of GluN2A KO mice. In UBC-CreERT/Grin2a^{flox/flox} mice, as shown in FIG. 1B, the expression of GluN2A protein was also significantly reduced in tamoxifen-treated mice, suggesting the successful establishment of GluN2A conditional knockout mice.

### 2. GluN2A KO Mice Exhibit Antidepressant-Like Behaviour and are not Dependent on Increased Spontaneous Mobility

Firstly, the spontaneous mobility of the mice was assessed by the open field test.

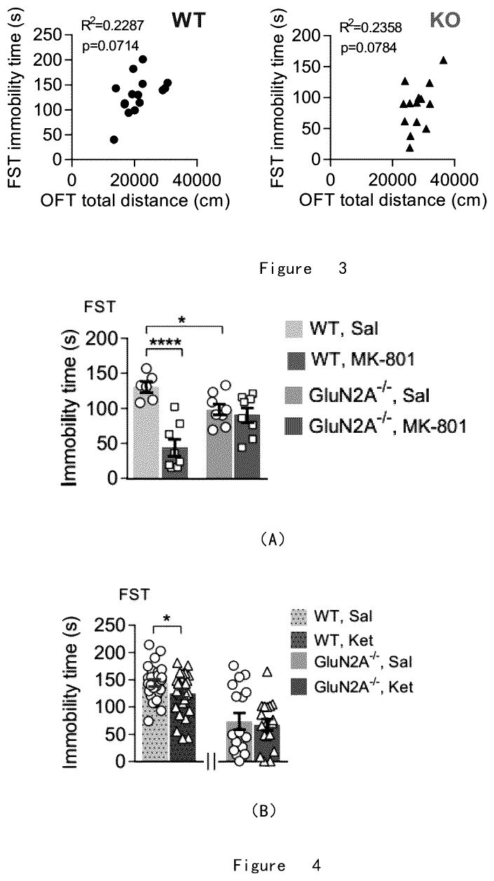

As shown in FIG. 2A, the travelled distance of GluN2A KO mice in the open field was significantly higher than that of WT mice, indicating an increase in the spontaneous mobility of GluN2A KO mice.

The depression-like behaviour of the mice was then analysed by the tail suspension tests and forced swim tests.

As shown in FIG. 2B, compared with WT mice, GluN2A KO mice showed significantly reduced immobility time in both tail suspension and forced swim tests, indicating that GluN2A KO mice exhibited antidepressant like behavior.

Followed by further analysis of whether there was a correlation between antidepressant behaviour and spontaneous mobility in GluN2A KO mice.

As shown in FIG. 3, there was no linear correlation between immobility time in the forced swim test and the traveled distance in the open field test in either WT or GluN2A KO mice, indicating that GluN2A KO mice exhibited antidepressant-like behaviour and were not dependent on increased spontaneous mobility.

### 3. Knockout of GluN2A Blocks the Antidepressant Effects of MK-801 and Ketamine

MK-801 and ketamine are non-competitive antagonists of NMDA receptors, which can induce rapid antidepressant like behavior in mice. WT and GluN2A KO mice were intraperitoneally injected with 10 mg/kg of MK-801 or ketamine, respectively, followed by the forced swim test.

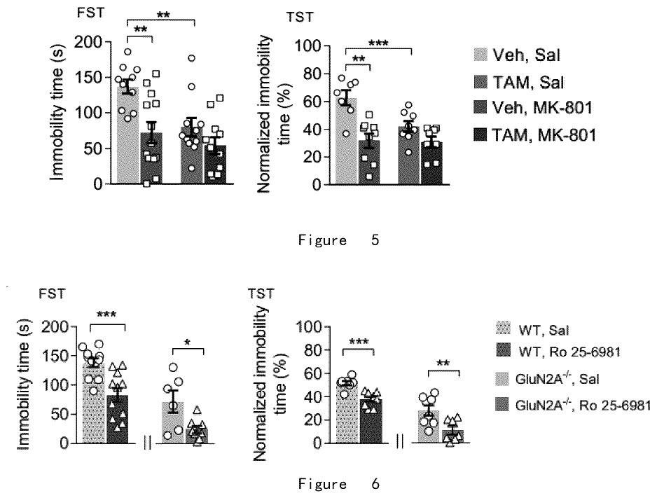

As shown in FIGS. 4A-B, 1 hour after intraperitoneal injection of MK-801 or ketamine, the immobility time of WT mice in the forced swim test was significantly reduced, indicating that MK-801 and ketamine can produce rapid antidepressant effects in WT mice. After being treated with the same MK-801 or ketamine, GluN2A KO mice showed comparable immobility time in forced swim test to the control group, indicating that MK-801 and ketamine cannot produce rapid antidepressant effects in GluN2A KO mice, which further suggested that the antidepressant effects of MK-801 and ketamine may be achieved by inhibiting GluN2A.

### 4. Knockout of Glu2A KO in Adult Mice can Lead to Antidepressant Like Behavior

Depression develops primarily during prime of life, so further study about whether the knockout of GluN2A in adulthood has antidepressant effects was conducted. 12 week-old UBC CreERT/Grin2a^{flox/flox} mice were intraperitoneally injected with Tamoxifen at a dose of 2 mg per mouse for 7 consecutive days, followed by tail suspension and forced swim tests after another week.

As shown in FIG. 5, compared with control mice, GluN2A conditional knockout mice showed significantly reduced immobility time in both tail suspension and forced swim tests, indicating that knockout of GluN2A in adulthood can lead to antidepressant like behavior in mice, and also suggesting that GluN2A is an important target for treating depression.

Furthermore, the immobility time of control mice was significantly reduced after administration of MK-801, whereas the immobility time of GluN2A conditional knockout mice was not altered after treatment with MK-801, suggesting that knockout of GluN2A in adult mice similarly masked the rapid antidepressant effect induced by MK-801.

5. Knockout of GluN2A does not Affect the Antidepressant Like Effects of GluN2B Selective Inhibitors

Ro 25-6981 is a known GluN2B selective antagonist and has also been found to induce antidepressant effects in mice.

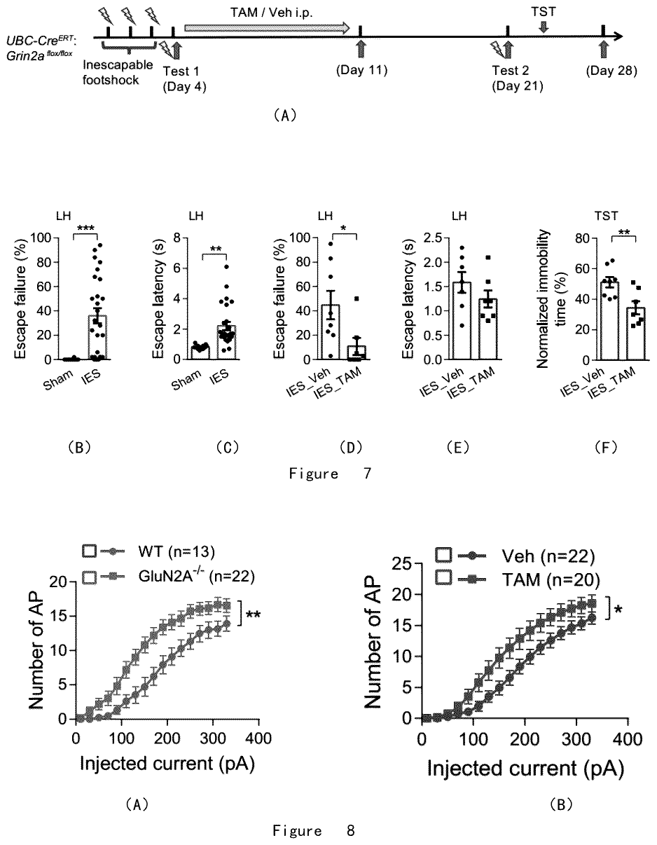

As shown in FIG. 6, WT mice treated with Ro 25-6981 (10 mg/kg) showed a significant decrease in immobility time in both forced swim test (A) and tail suspension test (B), compared to the control (Saline) group. Moreover, Ro 25-6981 also significantly reduced the immobility time of GluN2A KO mice in the forced swim test (A) and tail suspension test (B). This was different from the effect of the non-competitive antagonists MK-801 and ketamine of NMDA receptors in GluN2A KO as shown in the above results (FIG. 4), that is, “the knockout of GluN2A eliminated the rapid antidepressant effect induced by MK-801 and ketamine”. This suggested that the regulation on depression like behavior by GluN2A and GluN2B was achieved through different mechanisms.

### 6. GluN2A Knockout can Alleviate Depressive Like Behavior Induced by Learned Helplessness

To further evaluate the antidepressant effect of knockout of GluN2A, depression mice model was constructed using learned helplessness.

As shown in FIG. 7B-C, the UBC CreERT/Grin2a^{flox/flox} mice after experiencing inescapable footshocks showed a significant increase in escape failure rate and escape latency compared to the control group mice, indicating the successful establishment of depression mice model using learned helplessness.

Subsequently, depression model mice were treated with tamoxifen and control, respectively, and GluN2A was knocked out.

As shown in FIG. 7D, the escape failure rate significantly decreased after GluN2A knockout, indicating that Glu2A knockout alleviated depressive like behavior in depression mice.

In addition, the knockout effect of GluN2A was re-evaluated through tail suspension test. As shown in FIG. 7F, depression model mice showed significantly reduced immobility time in both tail suspension and forced swim tests after GluN2A knockout, indicating again that knockout of GluN2A alleviates depressant like behavior in mice, and also revealing that GluN2A is an important target for treating depression.

### 7. GluN2A Regulates Depressive Like Behavior by Regulating Neuronal Excitability

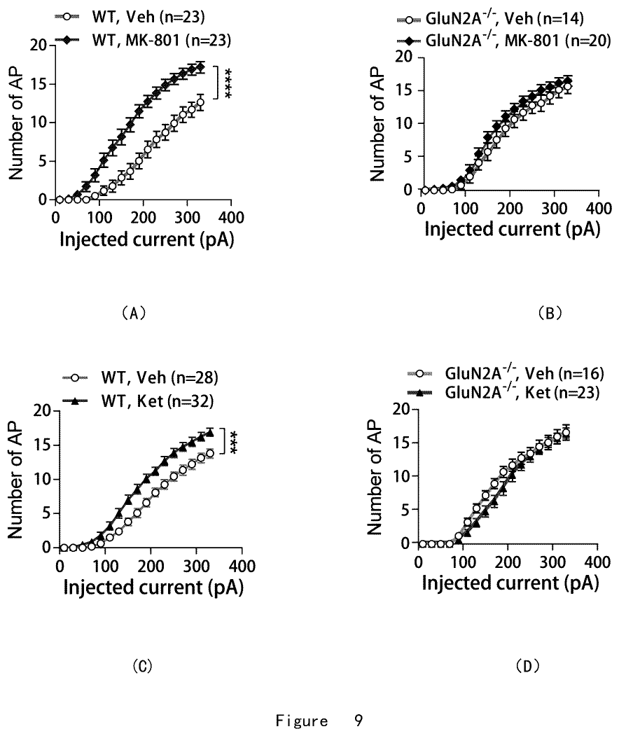

As shown in FIG. 8A, the number of action potentials generated by excitatory neurons in the hippocampal region of GluN2A KO mice was significantly higher than that of WT mice under the same depolarising current stimulation, suggesting that GluN2A knockout significantly increased the excitability of neurons in the hippocampal region. As shown in FIG. 8B, knockout of GluN2A in adult mice still increased the excitability of neurons in the hippocampal region.

Based on the above results, it was indicated that GluN2A regulates depressive like behavior by regulating the excitability of hippocampal neurons. To further confirm this viewpoint, hippocampal slices from WT mice and GluN2A KO were incubated with MK-801 and ketamine, respectively. As shown in FIG. 9, MK-801 and ketamine significantly increased the excitability of hippocampal neurons in WT mice, while GluN2A KO blocked the increase in excitability caused by MK-801 and ketamine. This was consistent with previous results, i.e., GluN2A knockout blocked the rapid antidepressant like behavior induced by MK-801 and ketamine in mice.

Behavioral experiments showed that GluN2A KO mice exhibit antidepressant like behavior. Knockout of GluN2A in adulthood can still alleviate depressive like behavior in depression model mice, and GluN2A knockout blocks the rapid antidepressant effects of MK-801 and ketamine.

Electrophysiological experiments showed that the excitability of excitatory neurons in the hippocampus of GluN2A KO mice increased, and the knockout of GluN2A eliminated the increase in neuronal excitability induced by MK-801 and ketamine.

8. GluN2B Regulates Antidepressant-Like Behaviour Through a Mechanism Different from GluN2A

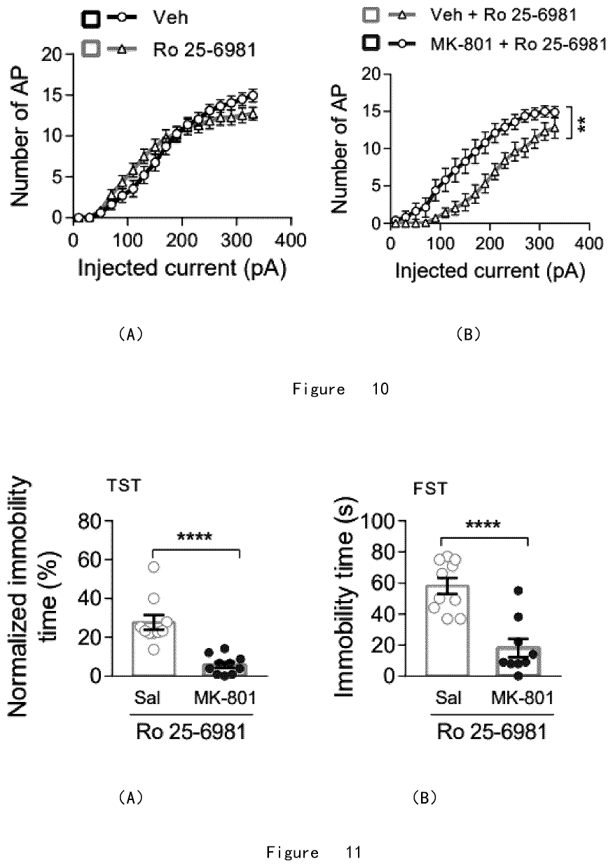

As shown in FIG. 10A, by using the same experimental method as above, it was found that the intrinsic excitability of excitatory neurons in the hippocampus of WT mice did not change after incubation with GluN2B selective inhibitor Ro 25-6981 (3 M) for 1-2 hours under the same depolarizing current stimulation, which was different from the effects of MK-801 and ketamine mentioned above (FIG. 9A, C). This result indicated that GluN2B was not involved in regulating the intrinsic excitability of excitatory neurons in the hippocampus.

As shown in FIG. 10B, acute hippocampal slices of WT mice were incubated with Ro 25-6981 while adding MK-801. MK-801 still induced an increase in intrinsic excitability of excitatory neurons in the hippocampus.

As shown in FIG. 11, behavioral experiments showed that when selectively inhibiting GluN2B in WT mice accompanied with treatment of MK-801, MK-801 can still induce rapid antidepressant like behavior. Combined with above results (FIG. 4A): “knockout of GluN2A blocked the antidepressant effect of MK-801”, it was again indicated that the rapid antidepressant effect of MK-801 was achieved by inhibiting GluN2A.

Therefore, the above results indicated that the selective inhibition of GluN2B leaded to rapid antidepressant effects through a completely different mechanism from GluN2A.

In summary, GluN2A subtype NMDA receptor has potentially important applications as a target in the treatment of depression, and the increased excitability of excitatory neurons can be used as a criterion for screening antidepressant drugs.

### DISCUSSION

The NMDA receptor, as a glutamatergic ion channel receptor, not only mediates excitatory glutamatergic transmission in the central nervous system, but also plays an integral role in synaptic plasticity, neuronal survival, learning and memory.

From the experimental results of the present invention, it can be concluded that antidepressant drugs such as MK-801 or ketamine failed to produce rapid antidepressant effects in GluN2A KO mice, while by selectively blocking GluN2B through pharmacology, MK-801 can still induce rapid antidepressant effects in WT mice. This suggests that the antidepressant effects of MK-801 and ketamine are achieved by inhibiting GluN2A. That is to say, GluN2A is a target of action of antidepressants. In other words, when a drug can inhibit GluN2A, it can produce antidepressant effects. Therefore, the present invention provides a use of GluN2A subtype NMDA receptors or GluN2A binding fragments for screening antidepressants.

Furthermore, in the depression animal model constructed through the learned helplessness paradigm, the conditional knockout of GluN2A in mice induced by Tamoxifen directly demonstrates that inhibiting GluN2A can alleviate, reverse/treat depression.

It should be noted that, unlike model mice that knock out the GluN2A gene in their genomes which can innately resist the development of depression (preventive effect and affect mouse brain development), the present invention demonstrates for the first time that inhibiting GluN2A can reverse/treat individuals suffer from depression developed in adulthood.

Thus, the present invention demonstrates that the GluN2A subtype NMDA receptor can be used as a target for treating depression, thus providing a new strategy for the development of antidepressant drugs. Further, provided is a medical use of GluN2A subtype NMDA receptor inhibitors for antidepression. The existing NMDA receptor antagonists that have been approved for marketing do not have subtype selectivity, which limits their use due to side effects. According to the present invention, specific GluN2A inhibitors can be developed for the treatment of depression, thereby reducing drug side effects.

All documents mentioned in the present invention are cited as references in this application, just as each document is individually cited as a reference. In addition, it should be understood that, after reading the above teaching content of the present invention, those skilled in the art can make various changes or modifications to the present invention, and these equivalent forms also fall within the scope defined by the appended claims of the present application.

1-3. (canceled)

4. A screening method for antidepressant drugs, wherein comprising steps of:
(i) providing GluN2A, a binding fragment thereof, and/or an NMDA receptor containing GluN2A;
(ii) contacting the drug candidate with GluN2A, the binding fragment thereof, and/or the NMDA receptor containing GluN2A; and
(iii) determining the inhibitory and/or binding ability of the drug candidate to GluN2A, the binding fragment thereof, and/or the NMDA receptor containing GluN2A; when the drug candidate possess the ability to inhibit and/or bind to GluN2A, the binding fragment thereof, and/or the NMDA receptor containing GluN2A, the drug candidate can be used as an antidepressant drug candidate.

5. The screening method according to claim 4, wherein, the method further comprises a step of:
(iv) testing the effect of the drug candidate screened out in step (iii) on excitatory neurons; when the inducement of antidepressant like behavior and/or increase in the intrinsic excitability of excitatory neurons by the drug candidate is detected, the drug candidate can be used as antidepressant drugs.

6-10. (canceled)

11. The screening method according to claim 4, wherein, the GluN2A has an amino acid sequence of SEQ ID No: 1 (human GluN2A) or an amino acid sequence of at least 80% identity to SEQ ID No: 1, preferably, GluN2A has an amino acid sequence with at least 85%, at least 90%, or at least 95% identity to SEQ ID No: 1.

12. The screening method according to claim 4, wherein, the GluN2A has an amino acid sequence of SEQ ID No: 2 (mouse GluN2A) or an amino acid sequence of at least 80% identity to SEQ ID No: 2, preferably, GluN2A has an amino acid sequence with at least 85%, at least 90%, or at least 95% identity to SEQ ID No: 2.

13. The screening method according to claim 4, wherein, the NMDA receptor containing GluN2A is an NMDA receptor containing two GluN1s and two GluN2A subunits, or an NMDA receptor containing at least one GluN2A subunit.

14. The screening method according to claim 4, wherein, the drug candidate is selected from the group consisting of: antibodies or binding fragments thereof, small molecule compounds or pharmaceutically acceptable salts thereof, and nucleic acids.

15. A screening device for antidepressant drugs, wherein, the device comprises a patch clamp device loaded with cells expressing GluN2A subtype NMDA receptors (such as HEK293, CHO, and Hela cells, etc.), wherein the device is configured to detect whether a candidate compound can reduce or block ion channel currents mediated by GluN2A subtype NMDA receptors;
or the device comprises:
(a) a first patch clamp device, wherein the first patch clamp device is a patch clamp device loaded with cells expressing GluN2A subtype NMDA receptors (such as HEK293, CHO, and Hela cells, etc.); and
(b) a second patch clamp device, wherein the second patch clamp device is a patch clamp device loaded with tissue sections of brain hippocampus from animals (such as rats, mice, etc.) or primary cultured neurons, wherein the device is configured to detect whether the candidate compound can enhance the intrinsic excitability of excitatory neurons.

16. A method for treating depression, wherein the method comprises a step of: administering an effective amount of GluN2A subtype NMDA receptor inhibitor to a subject suffering from depression, thereby treating depression.

17. The method according to claim 16, wherein, the GluN2A subtype NMDA receptor inhibitor is selected from the group consisting of: an antibody or a binding fragment thereof, a small molecule compound or a pharmaceutically acceptable salt thereof, and a nucleic acid.

18. The method according to claim 16, wherein, the GluN2A subtype NMDA receptor inhibitor is a compound selected from the group consisting of: 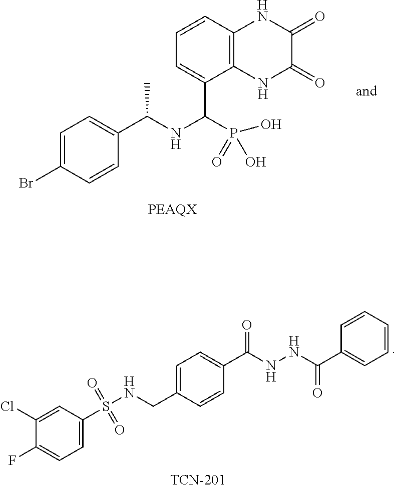 [SMILES: C[C@H](NC(c1cccc2[nH]c(=O)c(=O)[nH]c12)P(=O)(O)O)c1ccc(Br)cc1 ; O=C(NNC(=O)c1ccc(CNS(=O)(=O)c2ccc(F)c(Cl)c2)cc1)c1ccccc1]
or a pharmaceutically acceptable salt thereof.

19. The method according to claim 16, wherein, the GluN2A subtype NMDA receptor inhibitor is a monoclonal or polyclonal antibody thereof.

20. The method according to claim 16, wherein, the GluN2A subtype NMDA receptor inhibitor is not MK-801 or ketamine.
